(19) United States
(12) Patent Application Publication
(10) Pub. No.: US 2017/0188948 A1
(43) Pub. Date: Jul. 6, 2017

(54) WEARING METHOD AND APPARATUS THEREOF

(71) Applicant: Singular Wings Medical Co., Ltd., Hsinchu County (TW)

(72) Inventor: Hsien-Wen Chien, Hsinchu County (TW)

(73) Assignee: Singular Wings Medical Co., Ltd., Hsinchu County (TW)

(21) Appl. No.: 15/391,858

(22) Filed: Dec. 28, 2016

Related U.S. Application Data
(60) Provisional application No. 62/274,375, filed on Jan. 4, 2016.

(30) Foreign Application Priority Data
Nov. 28, 2016 (TW) 105139095

(51) Int. Cl. A61B 5/00; A61B 5/0205; A61B 5/11; A41D 1/00

(52) U.S. Cl. CPC A61B 5/6804; A41D 1/002; A61B 5/02055; A61B 5/11; A61B 5/0402

(57) ABSTRACT

The invention provides a wearing method and a wearing apparatus. The wearing method includes disposing a contact component on an inner side of a fabric to contact a user's skin; disposing an information capturing device on the fabric, wherein the information capturing device couples to the contact component; detecting at least one sensing information by the contact component and the information capturing device; and generating a prompt signal according to the at least one sensing information.

### CROSS REFERENCE TO RELATED APPLICATION

This application claims the priority benefits of U.S. provisional application Ser. No. 62/274,375, filed on Jan. 4, 2016, and Taiwan application serial no. 105139095, filed on Nov. 28, 2016. The entirety of each of the above-mentioned patent applications is hereby incorporated by reference herein and made a part of specification.

### BACKGROUND OF THE INVENTION

1. Field of the Invention

The invention relates to a wearing method and a wearing apparatus, and particularly relates to a wearing method and a wearing apparatus capable of measuring sensing information of a human body.

2. Description of Related Art

With the development of science and technology, conductive fibers are now applicable on clothes to offer an electromagnetic shielding effect. In addition, some in the textile industry propose to dispose conductive fibers on clothes to provide physiological information such as heart rate, respiration rate, allergic reaction, and the like. However, it is difficult to manufacture the clothes by directly weaving the conductive fibers in the clothes, and the physiological data obtained from the conductive fibers may not be sufficiently accurate. Thus, how to simply combine a physiological data sensor with normal clothes to achieve a more desirable sensing effect is an issue for people skilled in the art to work on.

### SUMMARY OF THE INVENTION

The invention provides a wearing method and a wearing apparatus, where a sensor is disposed in a fabric to provide a preferable effect of sensing physiological data.

The invention provides a wearing method, comprising: disposing a contact component on an inner side of a fabric to contact a user's skin; disposing an information capturing device on the fabric, the information capturing device being coupled to the contact component; detecting at least one sensing information by the contact component and the information capturing device; and generating a prompt signal based on the at least one sensing information.

According to an embodiment of the invention, the fabric includes a first surface and a second surface. The information capturing device comprises a third surface. The third surface contacts at least one of the first surface and the second surface, and the information capturing device is fixed onto the fabric by a friction force of the third surface with respect to the first surface or the second surface of the fabric.

According to an embodiment of the invention, the step of disposing the information capturing device on the fabric comprises: providing an accommodating space on the fabric by cutting an opening, and fixing the information capturing device to the accommodating space.

According to an embodiment of the invention, an adhering component is disposed on a third surface of the information capturing device, and the information capturing device is detachably fixed to the fabric by the adhering component.

According to an embodiment of the invention, the fabric comprises an elastic fiber, and the information capturing device is fixed between the fabric and the user's skin by a normal force applied by the elastic fiber to the user's skin.

According to an embodiment of the invention, the information capturing device further comprises a sensor, the sensor includes a physiological sensor, a movement sensor, and a global positioning system sensor. When a physiological sensing value of the physiological sensor exceeds a normal range and a motion sensing value of the movement sensor indicates that the user does not move or turn around within a predetermined time, a rescue request signal is transmitted based on location information of the global positioning system sensor.

The invention provides a wearing device, including a fabric, a contact component, and an information capturing device. The contact component is disposed on an inner side of the fabric to contact a user's skin. The information capturing device is disposed on the fabric and coupled to the contact component. The information capturing device detects at least one sensing information by the contact component, and generates a prompt signal based on the at least one sensing information.

According to an embodiment of the invention, the fabric comprises a first surface and a second surface, and the information capturing device is disposed on one of the first surface and the second surface of the fabric.

According to an embodiment of the invention, the fabric comprises a first surface and a second surface, and the information capturing device is disposed on the fabric and contacts the first surface and the second surface simultaneously.

According to an embodiment of the invention, the contact component comprises at least one of an electrode patch and a microphone.

According to an embodiment of the invention, the information capturing device is coupled to the contact component through a connection component.

According to an embodiment of the invention, the information capturing device comprises a sensor. The sensor comprises a physiological sensor and a movement sensor. The physiological sensor comprises at least one of an electrocardiogram sensor, a sound sensor, a temperature and moisture sensor, a perspiration pH value sensor, and an electromyography sensor. The movement sensor comprises at least one of an ultraviolet light sensor, an atmospheric pressure sensor, an air quality sensor, a geomagnetic sensor, a motion sensor, and a global positioning system sensor.

According to an embodiment of the invention, the sensor comprises a physiological sensor, a movement sensor, and a global positioning system sensor. When a physiological sensing value of the physiological sensor exceeds a normal range and a motion sensing value of the movement sensor indicates that the user does not move or turn around within a predetermined time, the information capturing device transmits a rescue request signal based on location information of the global positioning system sensor.

Based on the above, according to the wearing method and the wearing apparatus of the embodiments of the invention, the contact component may be disposed on an inner side of the fabric to contact the user's skin, and the information capturing device may be disposed on the fabric to be coupled to the contact component through the connection component. By independently disposing the fabric, the contact component, and the information capturing device, the contact component is able to accurately sense the physiological data and transmits the physiological data to the information capturing device. Thus, the information capturing device may output the corresponding prompt signal based on the physiological data.

In order to make the aforementioned features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a block diagram illustrating a wearing apparatus according to an embodiment of the invention.

FIGS. 2A and 2B are schematic views of a wearing apparatus according to an embodiment of the invention.

FIG. 3 is a flowchart illustrating a wearing method according to an embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

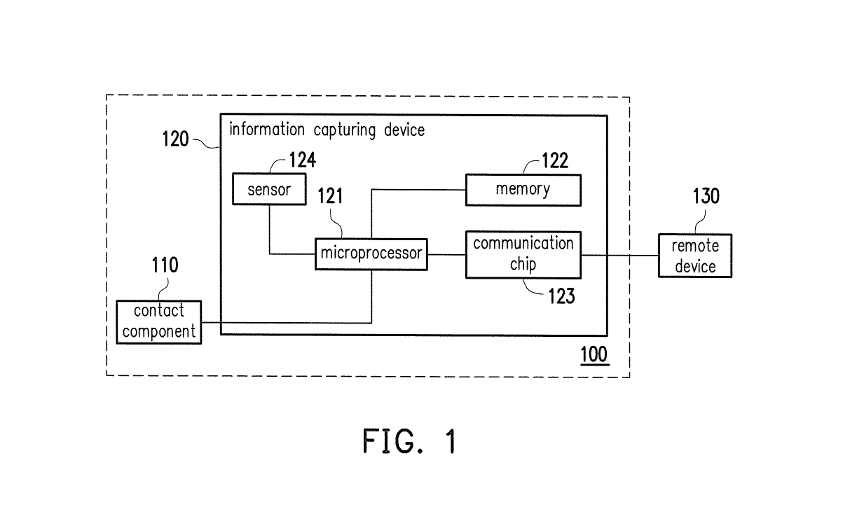

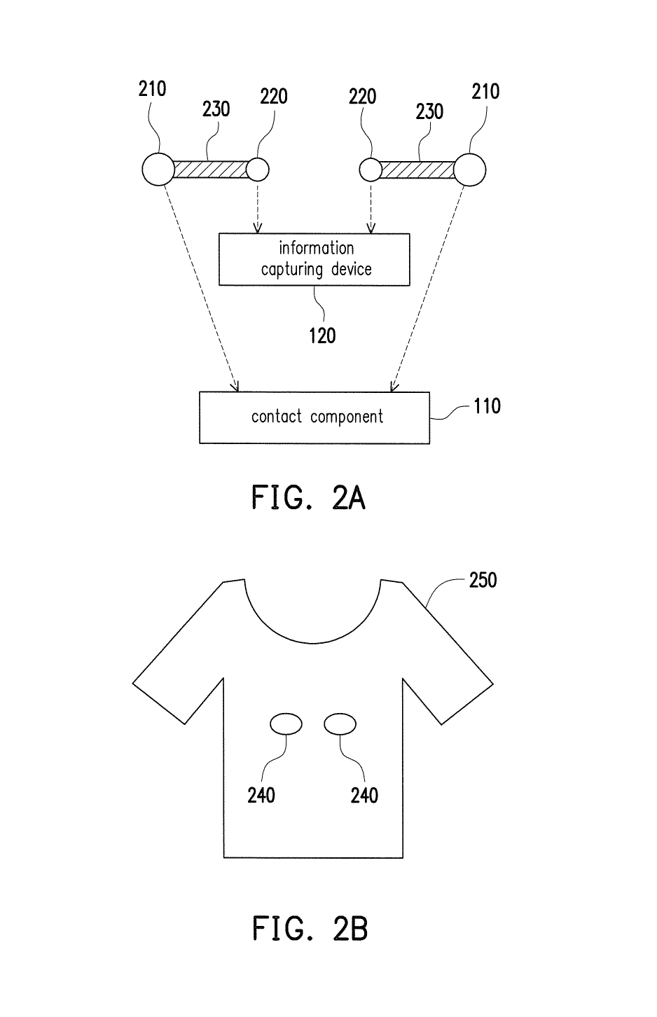

FIG. 1 is a block diagram illustrating a wearing apparatus according to an embodiment of the invention. FIGS. 2A and 2B are schematic views of a wearing apparatus according to an embodiment of the invention.

Referring to FIGS. 1, 2A, and 2B, a wearing device 100 includes a contact component 110, an information capturing device 120, and a fabric 250. The contact component 110 may be disposed on an inner side of the fabric 250 to contact the user's skin. The information capturing device 120 may be disposed on the fabric 250, such as on an inner surface, an outer surface, or between the inner and outer surfaces of the fabric 250 without contacting the user's skin. The information capturing device 120 includes a microprocessor 121, a memory 122, a communication chip 123, and a sensor 124. The information capturing device 120 is coupled to the contact component 110 through a connection component 230. The connection component 230 has a first end 210 and a second end 220. The first end 210 of the connection component 230 is coupled to the contact component 110 and the second end 220 is coupled to the information capturing device 120. In an embodiment, the fabric 250 may have a hole 240 with a cutting design to allow the connection component 230 to pass through the fabric 250. Consequently, the contact component 110 located on the inner surface of the fabric 250 may be coupled to the information capturing device 120 on the fabric 250 through the connection component 230.

In yet another embodiment, the information capturing device 120 may be fixed onto the fabric 250 by a friction force. Specifically, the fabric 250 may include a first surface and a second surface, and the information capturing device 120 may include a third surface (not shown). The third surface contacts at least one of the first surface and the second surface, and the information capturing device 120 is fixed onto the fabric 250 by a friction force of the third surface with respect to the first surface or the second surface of the fabric 250. It should be noted that a friction coefficient of the third surface of the information capturing device 120 may be greater than friction coefficients of other surfaces of the information capturing device 120 to allow the information capturing device 120 to be fixed on the fabric 250 by the friction force.

In another embodiment, the information capturing device 120 may be fixed onto the fabric 250 with tailoring. Specifically, an accommodating space (not shown) may be provided on the fabric 250 by cutting an opening, so as to fix the information capturing device 120 in the accommodating space.

In another embodiment, the information capturing device 120 may be fixed onto the fabric 250 by adhesion. Specifically, an adhering component (not shown) may be disposed on a surface of the information capturing device 120, and an adhered component (not shown) corresponding to the adhering component may be disposed at a specific position of the fabric 250. Consequently, the information capturing device 120 is detachably fixed onto the adhered component of the fabric 250 through the adhering component. For example, the adhering component and the adhered component may be a side of a velcro or any other detachable devices with an adhesion capability.

In yet another embodiment, the information capturing device 120 may be fixed onto the fabric 250 by an elastic force. Specifically, the fabric 250 includes an elastic fiber (not shown). The elastic fiber may be disposed to the whole fabric 250 or a portion of the fabric 250. Accordingly, when the user wears the fabric 250, the information capturing device 120 may be fixed between the fabric 250 and the user's skin by a normal force applied by the elastic fiber toward the user's skin.

In yet another embodiment, the information capturing device 120 may be disposed on one of the first surface and the second surface of the fabric 250 by the friction force, the adhesion, or the elastic force. Alternatively, the information capturing device 120 may be disposed on the fabric 250 and contact the first surface and the second surface of the fabric 250 simultaneously with tailoring.

The contact component 110 may include a physiological sensor configured to monitor a vital sign of the user, and may serve to monitor the user's physiological data such as electrocardiogram data, heart rate, pulse, sound, blood pressure, respiration rate, body temperature, or perspiration pH value, etc. To monitor the vital sign, the physiological sensor may directly contact the user's skin. For example, a dry-type or wet type electrode patch of an electrocardiogram (EKG) sensor, a sound sensing component (e.g., microphone), a dry-type or wet-type electrode patch of an electromyography (EMG) sensor, a photoplethysmogram (PPG) sensor, or a respiration pH value sensing component may be used to contact the user's skin. The contact component 110 may be formed by one or a combination of at least one electrical signal sensor (e.g., the EKG or EMG sensor) and a corresponding electrode patch or sound sensor (e.g., microphone).

The EKG or EMG sensor includes a conductive component (e.g., the dry-type or wet-type electrode patch), and may be connected to the information capturing device 120 through a connection component in various shapes and of various types. The sound sensing component may be a sound receiver, such as an MEMS microphone, an array microphone, or a piezoelectric microphone. Besides, the physiological sensor may also include a sensor such as a temperature and moisture sensor, a respiration pH value sensor, or the like.

It should be noted that sensing data generated by the contact component 110 may be directly transmitted to and processed by the microprocessor 121, or converted into digital data by an analog-to-digital converter (not shown) and then transmitted to and processed by the microprocessor 121.

The microprocessor 121 may be a digital signal processor (DSP), a programmable controller, a microcontroller, an application specific integrated circuit (ASIC), a programmable logic device (PLD), or other similar devices. The microprocessor 121 may perform sampling, quantifying, noise-suppressing, or other data preprocessing on physiological or movement data of a human body. The memory 122 and the communication chip 123 may be coupled to the microprocessor 121. The memory 122 may be any random access memory or non-volatile memory. The communication chip 123 may be a communication chip compatible with Bluetooth transmission, WIFI transmission, or other wireless communication transmission.

The sensor 124 may be coupled to the microprocessor 121 and may include a movement sensor (e.g., at least one of an ultraviolet light sensor, an atmospheric pressure sensor, an air quality sensor, a geomagnetic sensor, a motion sensor, and a global positioning system sensor, wherein the motion sensor may be a three-axis accelerometer or a gyroscope). The movement sensor may serve to detect the user's movement information, such as height, speed, distance, or the like. It should be noted that the movement sensor does not need to directly contact the user's skin, thereby offering the user a more comfortable wearing experience. Also, the ultraviolet light sensor may be disposed to an external part of the fabric 250 to sense an intensity of ultraviolet light.

In an embodiment, when the microprocessor 121 determines that a physiological sensing value of the physiological sensor exceeds a normal range, and a motion sensing value of the movement sensor indicates that the user does not move or turn around within a predetermined time, the microprocessor 121 may transmit a rescue request signal based on location information of the global positioning system sensor. Specifically, when physiological data, such as pulse, blood pressure, or the like, indicates an abnormal state, and the accelerometer or the gyroscope detects that the human body does not move or turn around within a prescribed time, the microprocessor 121 may determine that the user may lose consciousness due to a disorder, and thus transmit a rescue request signal to the emergency personnel so as to rescue the user based on the location of the rescue request. Accordingly, a mortality rate of users living alone and collapsing at home without being noticed may be reduced.

When the sensor 124 and the contact component 110 detect sensing information, the information capturing device 120 may receive the sensing information and generate a prompt signal based on the sensing information. The prompt signal may prompt the user by lighting, sound, electricity, vibration, or the like. More specifically, based on the sensing information detected by the sensor 124 and the contact component 110, the microprocessor 121 may analyze a heart beat rhythm and determine heart-related diseases based on an EKG waveform, and calculate body age, pressure index, and state of emotion (e.g., nervous, excited, scared, pressured, etc.). The microprocessor 121 may further analyze a blood pressure index to determine an index (e.g., low blood pressure, normal, high normal, mild high blood pressure, intermediate high blood pressure, severe high blood pressure, etc.). Lastly, the microprocessor 121 may generate a corresponding prompt signal based on the result of analysis, so as to provide information for improvement.

The sound sensor may analyze and find out issues relating to sounds of internal organs such as heart, lung, and intestine, and sounds of neck and throat. The microprocessor 121 may generate a corresponding prompt signal based on the result of analysis, so as to provide information for prevention.

The temperature and moisture sensor may serve to analyze a core temperature (e.g., body temperature or environmental temperature) and provide relevant information for prevention. For example, a persistently high body temperature may indicate infection or inflammation of a body part or a rheumatic disease that requires immediate medical examination. Alternatively, a persistently low body temperature may indicate severe infection of a body part causing worsening of blood circulation that also requires immediate medical examination. Therefore, the microprocessor 121 may generate a corresponding prompt signal based on the result of analysis to prompt the user to take a medical examination.

The respiration pH value sensor may analyze the data relating to electrolyte and pH value. For example, an electrolyte level may indicate whether one is dehydrated. When someone is dehydrated, the microprocessor 121 may generate a corresponding prompt signal to remind the user to take more water.

The ultraviolet light sensor may analyze an ultraviolet light index, and determine whether the index is at a low, moderate, high, excessive, or dangerous level. When the ultraviolet light index indicates a level of high or above, the microprocessor 121 may generate a corresponding prompt signal to remind user to reinforce sun block protection.

The atmospheric pressure sensor may detect an atmospheric pressure and serve as an altimeter. Information from the atmospheric pressure sensor may serve for vertical navigation, and is applicable in local weather forecasting or warning of abnormal weather. When there is a sudden change of atmospheric pressure, the microprocessor 121 may generate a corresponding prompt signal to remind user to be aware whether the weather is abnormal.

The air quality sensor may detect a level of local air pollution for regional air quality forecasting. When the air quality is drastically worsened, the microprocessor 121 may generate a corresponding prompt signal to remind user to refrain from outdoor activities.

The electromyography sensor may detect a muscle surface current as an index of muscle usage or nerve conduction. The geomagnetic sensor may detect a geomagnetic direction to identify a direction of the device. A motion sensor may determine acceleration, deceleration, and rotational angle of a movement of the device, so as to determine all movement states of the device, and may serve for motion detection and fall-down detection of the user.

Besides, the information capturing device 120 may further transmit the sensing information to a remote device 130 for recording through the communication chip 123, and may transmit the prompt signal to the remote device 130 for display or warning. The remote device 130 may be a personal computer, a server, a smartphone, a tablet computer, or a mobile device of other types.

The connection component 230 includes a conductive transmissive material, and may transmit the signal received by the contact component 110 to the information capturing device 120 through wired transmission. The connection component 230 may be a conductive wire such as a signal line or a conductive component, and may serve as a bridge of signal transmission between the information capturing device 120 and the contact component 110.

The fabric 250 may be designed to be a fitting or non-fitting wearing fabric based on tailoring or changing the material of the fabric, and the fabric 250 may also be designed to fix the contact component 110 or the information capturing device 120 based on a fitting design of the fabric 250. In addition, the fabric 250 and the information capturing device 120, the contact component 110, and the connection component 230 may be presented independently or in combination to achieve a smart fabric.

It should be noted that the information capturing device 120, the contact component 110, and the connection component 230 may be connected in fixed or detachable manner, and may be designed to be easy to install through embedding and unloading. In an embodiment, the information capturing device 120, the contact component 110, and the connection component 230 may be connected in fixed. The contact component 110 is located on the inner surface of the fabric 250, and the information capturing device 120 may be located on the inner surface or the outer surface of the fabric 250. In the embodiment, the connection component 230 may be disposed in the fabric physically (e.g., by tailoring, an opening, a friction force, an elastic force, adhesion, or the like), so that the contact component 110 contacts human skin, and the information capturing device 120 is electrically connected with the connection component 230 by being exposed to the outer surface of the fabric 250 or covered in the inner surface of the fabric 250.

In another embodiment, the information capturing device 120, the contact component 110, and the connection component 230 may be connected in detachable ma er. The contact component 110 is located on the inner surface of the fabric 250, and the information capturing device 120 may be located on the outer surface of the fabric 250. In yet another embodiment, the contact component 110 and the information capturing device may be located on the inner surface of the fabric 250. In the embodiment, the connection component 230 may be disposed in the fabric physically (e.g., by tailoring, an opening, a friction force, an elastic force, adhesion, or the like), so that the contact component 110 contacts human skin, and the information capturing device 120 is electrically connected with the connection component 230 by being exposed to the outer surface of the fabric 250 or covered in the inner surface of the fabric 250.

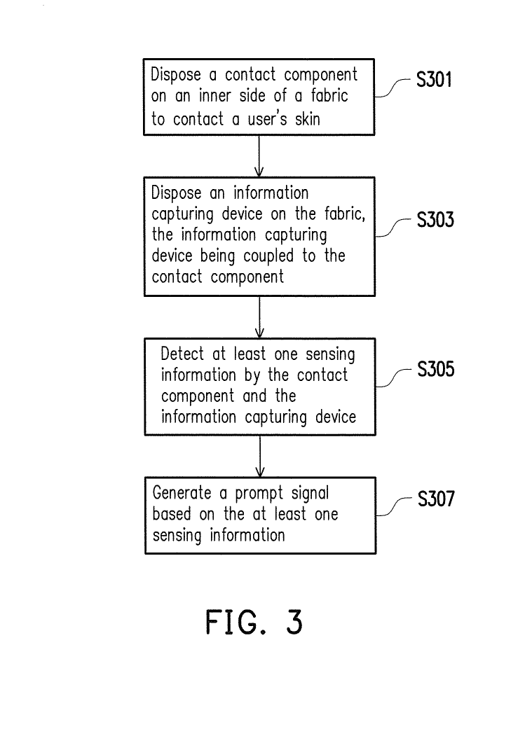

FIG. 3 is a flowchart illustrating a wearing method according to an embodiment of the invention.

Referring to FIG. 3, at Step S301, the contact component is disposed on the inner side of the fabric.

At Step S303, the information capturing device is disposed on the fabric. The information capturing device is coupled to the contact component. Disposing the information capturing device on the fabric indicates that the information capturing device is disposed on the inner surface or the outer surface of the fabric by tailoring, an opening, a friction force, an elastic force, adhesion, or the like.

At Step S305, at least one sensing information is detected by the contact component and the information capturing device. The sensing information may include physiological information or movement information.

At Step S307, the prompt signal is generated based on the at least one sensing information. The user may realize the current physiological or movement state based on the prompt signal, so as to adjust a movement rhythm or seek help when in an undesirable physiological state.

In view of the foregoing, the embodiments of the invention provide a wearing method and a wearing apparatus. The contact component and the information capturing device are connected by tailoring design of the fabric, and the user's physiological state and movement state are detected by the physiological sensor and the movement sensor, so as to output the corresponding prompt signal. The contact component may be disposed on the inner surface of the fabric to contact the user to obtain the relevant physiological data. The information capturing device may be disposed on the outer surface or the inner surface of the fabric without directly contacting the human body, so as to make the user feel more comfortable. In addition, the connection between the contact component and the information capturing device may be connected in fixed or detachable manner, so as to make the design more flexible.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A wearing method, comprising:
disposing a contact component on an inner side of a fabric to contact a user's skin;
disposing an information capturing device on the fabric, wherein the information capturing device is coupled to the contact component;
detecting at least one sensing information by the contact component and the information capturing device; and
generating a prompt signal based on the at least one sensing information.

2. The wearing method as claimed in claim 1, wherein the fabric comprises a first surface and a second surface, the information capturing device comprises a third surface, the third surface contacts at least one of the first surface and the second surface, and the information capturing device is fixed onto the fabric by a friction force of the third surface with respect to the first surface or the second surface of the fabric.

3. The wearing method as claimed in claim 1, wherein the step of disposing the information capturing device on the fabric comprises: providing an accommodating space on the fabric by cutting an opening, and fixing the information capturing device to the accommodating space.

4. The wearing method as claimed in claim 1, wherein an adhering component is disposed on a third surface of the information capturing device, and the information capturing device is detachably fixed to the fabric by the adhering component.

5. The wearing method as claimed in claim 1, wherein the fabric comprises an elastic fiber, and the information capturing device is fixed between the fabric and the user's skin by a normal force applied by the elastic fiber to the user's skin.

6. The wearing method as claimed in claim 1, wherein the information capturing device further comprises a sensor, the sensor comprises a physiological sensor, a movement sensor, and a global positioning system sensor, wherein when a physiological sensing value of the physiological sensor exceeds a normal range and a motion sensing value of the movement sensor indicates that the user does not move or turn around within a predetermined time, a rescue request signal is transmitted based on location information of the global positioning system sensor.

7. A wearing apparatus, comprising:
a fabric;
a contact component, disposed on an inner side of the fabric to contact a user's skin;
an information capturing device, disposed on the fabric and coupled to the contact component, wherein the information capturing device detects at least one sensing information by the contact component, and generates a prompt signal based on the at least one sensing information.

8. The wearing device as claimed in claim 7, wherein the fabric comprises a first surface and a second surface, and the information capturing device is disposed on one of the first surface and the second surface of the fabric.

9. The wearing device as claimed in claim 7, wherein the fabric comprises a first surface and a second surface, and the information capturing device is disposed on the fabric and contacts the first surface and the second surface simultaneously.

10. The wearing device as claimed in claim 7, wherein the contact component comprises at least one of an electrode patch and a microphone.

11. The wearing device as claimed in claim 7, wherein the information capturing device is coupled to the contact component through a connection component.

12. The wearing device as claimed in claim 7, wherein the information capturing device comprises a sensor, the sensor comprises a physiological sensor and a movement sensor, the physiological sensor comprises at least one of an electrocardiogram sensor, a sound sensor, a temperature and moisture sensor, a perspiration pH value sensor, and an electromyography sensor, and the movement sensor comprises at least one of an ultraviolet light sensor, an atmospheric pressure sensor, an air quality sensor, a geomagnetic sensor, a motion sensor, and a global positioning system sensor.

13. The wearing device as claimed in claim 7, wherein the sensor comprises a physiological sensor, a movement sensor, and a global positioning system sensor, wherein when a physiological sensing value of the physiological sensor exceeds a normal range and a motion sensing value of the movement sensor indicates that the user does not move or turn around within a predetermined time, the information capturing device transmits a rescue request signal based on location information of the global positioning system sensor.
